(19) United States
(12) Patent Application Publication
(10) Pub. No.: US 2020/0323477 A1
(43) Pub. Date: Oct. 15, 2020

(54) BALANCE TESTING APPARATUS

(71) Applicant: THE UNIVERSITY OF WESTERN ONTARIO, London (CA)

(72) Inventors: Jacob MacKenzie REEVES, London (CA); Bartosz SLAK, Windsor (CA); Maryanne Man-Yan SIU, Toronto (CA); Ibrahim MARWA, London (CA); Mahmoudreza RAMIN, London (CA)

(21) Appl. No.: 16/849,502

(22) Filed: Apr. 15, 2020

Related U.S. Application Data
(60) Provisional application No. 62/834,240, filed on Apr. 15, 2019.

(51) Int. Cl. A61B 5/00; A61B 5/107

(52) U.S. Cl. CPC A61B 5/4023; A61B 5/7405; A61B 5/742; A61B 5/1072

(57) ABSTRACT

A balance testing apparatus for testing balance of a test subject having a stance foot and an excursion foot includes a mat, at least one elongate reference line and an electronic measuring device. The mat has a stance foot orientation guide for receiving the stance foot. The at least one elongate reference line extends radially from the stance foot orientation guide. The electronic measuring device is designed for measuring the distance between the stance foot and the excursion foot when the excursion foot is on or near the reference line. A method of balance testing using the balance testing apparatus includes the steps of: measuring a first predetermined parameter having the stance foot in a first predetermined position; storing the data; repeating the steps with the stance foot in a different predetermined position; and evaluating the data to obtain a balance test protocol score.

### FIELD OF THE DISCLOSURE

This disclosure relates to testing apparatus and in particular balance testing apparatus and methods of using same.

### BACKGROUND

Balance tests are used for a variety of purposes and in a variety of different contexts. For example, balance tests may be used after a physical injury or a brain injury to determine impact of the injury and monitor progress after the injury.

A common balance test that is used is the Star Excursion Balance Test (SEBT) which is a quantitative test to assess lower limb injuries, such as ankle sprains. In this assessment, a patient is directed to stand with one foot located at the intersection of eight lines that radiate outward at equal intervals around the patient. The patient must then perform an excursion along each direction independently and lightly touch the toe to the floor as far away from their body as they can without compromising their balance. The practitioner then measures the excursion distance; provided the patient kept their hands on their hips, did not compromise their form or place substantial weight on their excursion foot. The excursion measurement is then repeated in each of the eight directions. The excursion directions are typically indicated by tape that a practitioner places on the floor. This test has largely remained a manual tape-based balance assessment since its inception.

To combat the time burden associated with the SEBT a variation of the test in which the patient only performs excursions in 3 equally spaced directions has been developed. This is typically referred to as the Y-balance test. Some variants of the Y-balance test have transitioned to a system where the patient pushes wooden blocks along each excursion direction in order to indicate the maximum distance of excursion. Although simpler, this Y-balance assessment has still not been widely adopted in clinical practice because of the same spatial and manual burdens associated with the traditional SEBT assessment.

There are alternative tests to assess balance and lower-limb injury such as: non-standardized movement observation, Tinetti balance assessment (sit down and rising balance test), and walking balance assay.

Accordingly, it would be advantageous to provide a balance testing apparatus that is quick to set up and easy to use.

### SUMMARY

A balance testing apparatus for testing balance of a test subject having a stance foot and an excursion foot includes a mat, at least one elongate reference line and an electronic measuring device. The mat has a stance foot orientation guide for receiving the stance foot. The at least one elongate reference line extends radially from the stance foot orientation guide. The electronic measuring device is designed for measuring the distance between the stance foot and the excursion foot when the excursion foot is on or near the reference line.

The electronic measuring device may be a haptic distance sensor that is located in registration with at least one elongate reference line.

The electronic measuring device may be one of a lidar, capacitive, resistive, ultrasonic, time-of-flight or pressure or temperature sensitive device that measures distance.

The balance testing apparatus may further include an electronic data storage device and a visual display operably connected to the electronic measuring device for storing and displaying the measured distance.

The visual display may display diagrams to help guide the subject through the testing responsive to the subject position in the testing.

The electronic data storage device may be any one of a mobile device, a personal computer, a tablet, the cloud or a combination thereof.

The balance testing apparatus may further include an audio device operably connected to the electronic data storage device.

The audio device may provide audio instructions.

The audio instructions may be responsive to information collected from the electronic measuring device.

The stance foot orientation guide may include a central reference point for indicating the position of the stance foot.

The stance foot orientation guide may further include a plurality of radial spokes. The plurality of radial spokes may be 8 equally spaced radial spokes.

The stance foot orientation guide may be colour coded to differentiate between the position for a right stance foot and a left stance foot.

The stance foot orientation guide may further include a plurality of LED lights. The plurality of LED lights may be activated responsive to the position of the stance foot to indicate the next orientation for the stance foot.

The balance testing apparatus may further include a center of gravity platform. The center of gravity platform may include a transparent surface that is positioned over the orientation guide. The center of gravity platform may be an integral part of the mat. The center of gravity platform may include at least one load cell for determining the center of gravity of the stance foot of the subject. The at least one load cell may be a plurality of load cells. The center of gravity platform may determine the weight of the subject. The center of gravity platform may determine the percentage of the weight of the subject that is offloaded when the excursion foot of the subject touches the ground.

The balance testing apparatus may be collapsible.

A method of balance testing using the balance testing apparatus includes the steps of: measuring a first predetermined parameter having the stance foot in a first predetermined position; storing the data; repeating the steps with the stance foot in a different predetermined position; and evaluating the data to obtain a balance test protocol score.

The method of balance testing may further include the step of determining the center of gravity on the stance foot in the different positions during the plurality of step a).

The method of balance testing may further include the percentage of the weight of the subject that is offloaded when the excursion foot of the subject touches the ground.

The balance test apparatus may include a program that provides instructions to the test subject responsive to the location of the stance foot.

The balancing testing apparatus may include LED lights and the lights indicate the orientation of the stance foot.

The pre-recorded audio instructions may be provided responsive to the location of the stance foot.

A flexible sensing strip for use in association with a balance testing mat for testing balance of a test subject having a stance foot and an excursion foot. The flexible sensing strip includes an electronic measuring device for measuring the distance between the stance foot and the excursion foot when the excursion foot is on or near the reference line having a distance sensor; a display operably connected to the elongate distance sensor; and a speaker operably connected to the elongate distance sensor.

The elongate distance sensor may be sandwiched between a top surface and a bottom surface. The top surface and bottom surface may be water resistant. The bottom surface may include an adhesive.

The distance sensor may be a haptic distance sensor.

Further features will be described or will become apparent in the course of the following detailed description.

### BRIEF DESCRIPTION OF THE DRAWINGS

The embodiments will now be described by way of example only, with reference to the accompanying drawings, in which:

FIG. 1 is a top view of a balance testing apparatus;

FIG. 2 is an enlarged top view of the stance foot zone of the balance testing apparatus of FIG. 1;

FIG. 3 is an assembly view of the balance testing apparatus of FIG. 1;

FIG. 4 is a top view of an alternate balance testing apparatus similar to that shown in FIG. 1 but also including a center-of-gravity platform in the stance foot zone;

FIG. 5 is an enlarged top view of the center-of-gravity platform of FIG. 4;

FIG. 6 is an assembly view of the center-of-gravity platform of FIGS. 4 and 5;

FIG. 7 is a schematic view of the control system for the balance testing apparatus 10 of FIG. 1;

FIGS. 8A to 8G are a pictorial representation of steps used for testing balance of a test subject;

FIG. 8A choose the stance foot;

FIG. 8B place stance foot in the stance foot zone;

FIG. 8C stand up straight with hands on hip;

FIG. 8D reach the excursion foot as far as possible along the excursion line in the excursion zone of the balance testing apparatus;

FIG. 8E lightly tap the toe of the excursion foot on the excursion line;

FIG. 8F identify if the tap is successful;

FIG. 8G rotate stance foot and repeat;

FIG. 9A to 9H are top views of the stance foot zone showing pictorial representations of the stance foot and the direction of reach for the excursion foot;

FIG. 9A is the first position with the stance foot being the right foot and being in line with the excursion line in the excursion zone and showing the reach of the excursion foot in front of the stance foot;

FIG. 9B is the second position with the stance foot positioned 45 degrees from the first position and the excursion foot extending to the side of the stance foot;

FIG. 9C is the third position with the stance foot positioned 90 degrees from the first position and the excursion foot extending to the side of the stance foot;

FIG. 9D is the fourth position with the stance foot positioned 135 degrees from the first position and the excursion foot extending backwards past the stance foot;

FIG. 9E is the fifth position with the stance foot positioned 180 degrees from the first position and the excursion foot extending behind the stance foot;

FIG. 9F is the sixth position with the stance foot positioned 225 degrees from the first position and the excursion foot extending behind the stance foot;

FIG. 9G is the seventh position with the stance foot positioned 270 degrees from the first position and the excursion foot extending behind the stance foot;

FIG. 9H is the eighth position with the stance foot positioned 315 degrees from the first position and the excursion foot extending backwards past the stance foot;

FIG. 10 is an alternate embodiment of the balance testing apparatus similar to that shown in FIG. 1 but further including a plurality of LEDs;

FIG. 11 is another alternate embodiment of the balance testing apparatus similar to that shown in FIG. 1 but including a Line-of-Sight device 35;

FIG. 12 is an assembly view of a flexible sensing strip;

FIG. 13 is a top view of the flexible sensing strip of FIG. 12;

FIG. 14 is a partial rolled top view of the flexible sensing strip of FIGS. 12 and 13; and

FIG. 15 is a top view of the flexible sensing strip of FIGS. 12 to 15 in association with an example of a prior art Star Excursion Balance Test mat.

### DETAILED DESCRIPTION

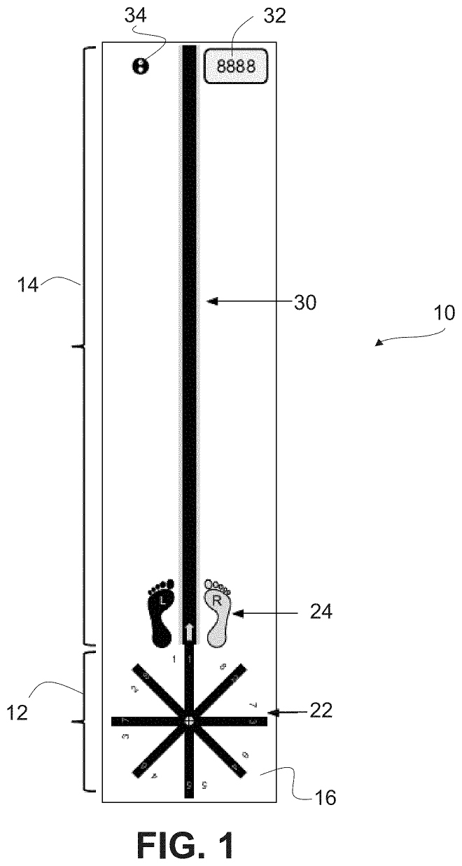

Referring to FIG. 1, a balance testing apparatus is shown generally at 10. The balance testing apparatus 10 includes a stance foot zone 12 and an excursion zone 14.

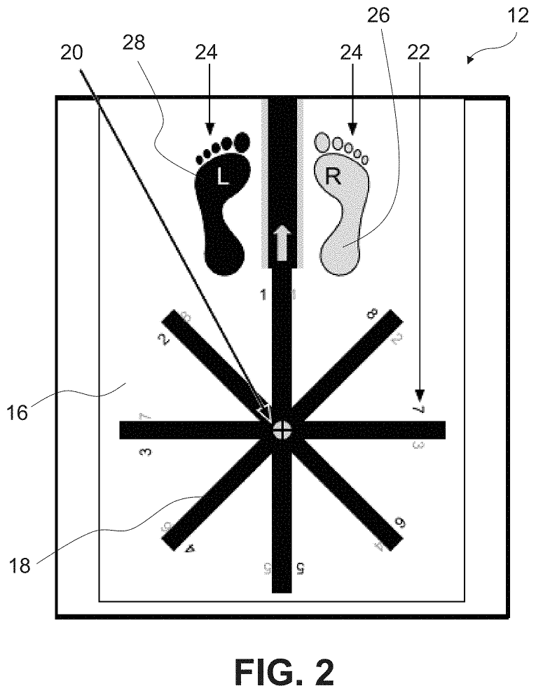

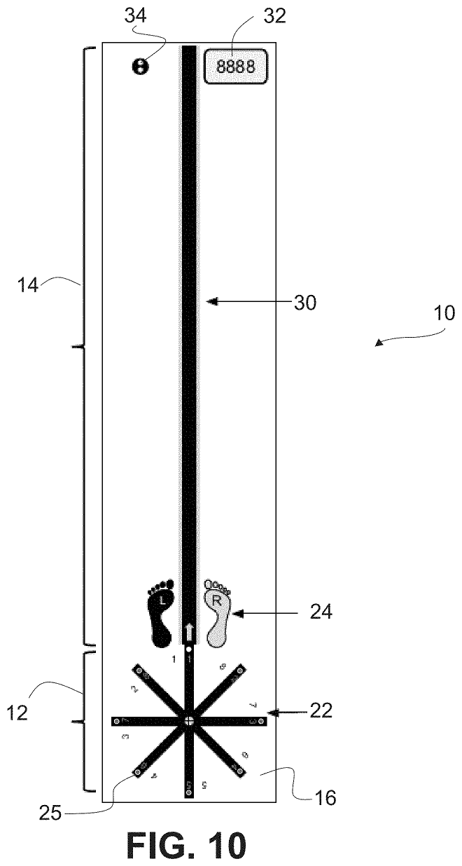

The stance foot zone 12 as best seen in FIG. 2 includes a stance foot orientation guide 16. The stance foot orientation guide 16 includes a plurality of radial spokes 18 that extend outwardly from a central reference point 20. The central reference point 20 identifies the position for the stance foot. In the embodiment shown herein the plurality of radial spokes 18 include eight (8) equally spaced radial spokes. It will be appreciated by those skilled in the art that the eight radial spokes are by way of example only and that the number of radial spokes could be changed by the user. However, eight radial spokes correspond to the standard SEBT currently in use. As the subject moves through the balance test the orientation of the stance foot is moved to positions aligned with the radial spokes 18. The stance orientation numbers 22 indicate the order for positioning of the stance foot around the radial spokes 18. The stance orientations numbers 22 may be positioned on or near the radial spokes 18. A stance foot colour guide 24 provides different colours for the right foot 26 and the left foot 28. The colour stance orientation numbers 22 correspond to the stance foot colour guide 24. As an alternative the balance testing apparatus 10 may also include LED lights 25 positioned around the radial spokes 18, as shown in FIG. 10. The LED lights 25 are configured to be activated responsive to the position of the stance foot to indicate the next orientation for the stance foot.

As best seen in FIG. 1 the excursion zone 14 has a touch sensitive zone which is an elongate reference line 30 that extends along the length of the excursion zone and extends outwardly from the central reference point 20 of the stance foot orientation guide 16.

The balancing test apparatus 10 also includes a display 32 for displaying the distance measurement and a speaker 34 that may provide instructions to the subject as they go through the balancing test. The speaker may be used to provide a beep when the distance for the excursion foot is recorded and the subject will then know that they can move on to the next position. As well, the speaker may be configured to provide audio instructions that are responsive to information collected from the electronic measuring device. The audio instructions may be in the form of pre-recorded verbal feedback, beeps or any other form of audio feedback. Similarly, the display 32 may be used to provide visual instructions that are responsive to information collected from the electronic measuring device.

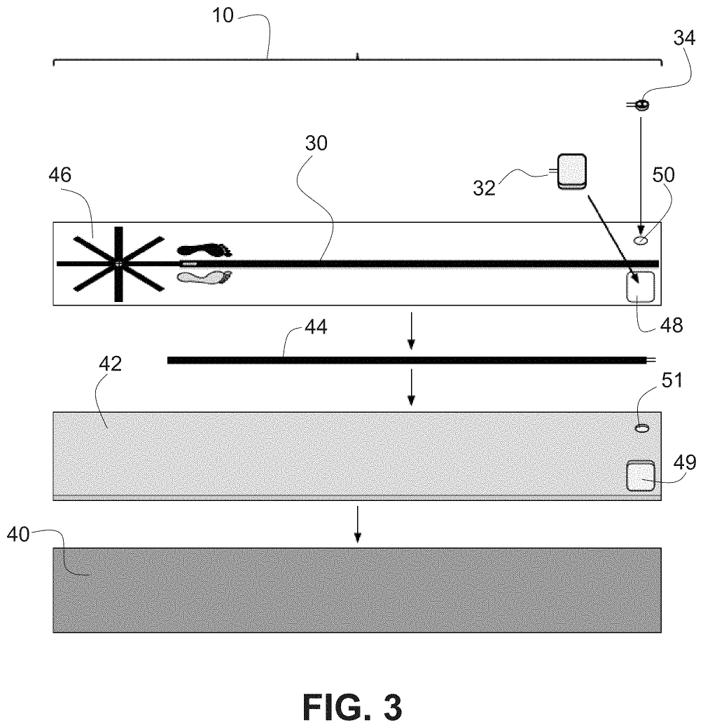

Referring to FIG. 3, the balancing test apparatus 10 includes a plurality of layers. The bottom grip layer 40 is a grip surface designed to minimize the likelihood of the balancing test apparatus 10 slipping on the floor. The bottom grip layer 40 is attached to a base layer 42. The base may be a rigid base or alternatively be a foam or other less rigid material. In some embodiments the base layer may be removed. For the less rigid alternatives the balancing test apparatus 10 may be rollable. A distance sensor 44 is attached to the rigid base layer 42. The distance sensor 44 may be a haptic distance sensor. A top layer 46 is a durable surface with the stance foot orientation guide 16 and the elongate reference line 30 thereon. The haptic distance sensor 44 is in registration with the elongate reference line 30. The distance display 32 and the speaker 34 are attached through holes 48 and 50 respectively to the base 42. Similarly holes 49 and 51 may be formed in the rigid base layer 42 in registration with holes 48 and 50. Alternatively holes 49 and 51 within the base 42 may be a cavity or simply a registration area on which the necessary electronics are placed. It will be appreciated by those skilled in the art that there are a variety of different methods of applying the foot orientation guide 16 and the elongate reference line 30 to the rigid base layer 42. It will be appreciated by those skilled in the art that the bottom grip layer 40 may be a separate layer or a surface treatment applied to the rigid base layer 42.

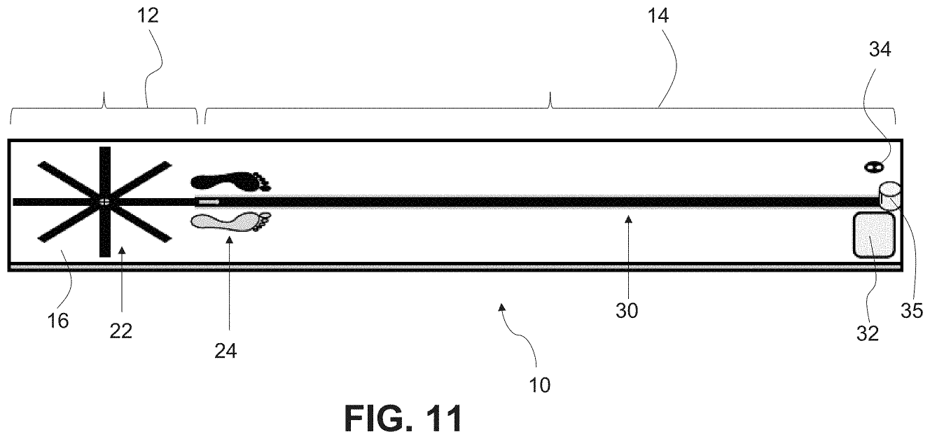

It will be appreciated by those skilled in the art that the balance testing apparatus 10 may have a number of variations. For example, the haptic sensor 44 may be a lidar, capacitive, resistive, ultrasonic, time-of-flight or pressure sensitive device that measures distance. Ultrasonic and Lidar work on the principle of Time-of-Flight, measuring how long it takes a wave to propagate to the excursion foot and back. An alternate embodiment which uses a Time-of-Flight device 35 rather than the haptic distance sensor 44 is shown in FIG. 11. The Line-of-Sight module 35 sits above the top surface so that the wave (sound or light) can be tripped by the excursion foot. Resistive sensors change the electrical resistance detected depending on how far along the sensor the touch is detected. Capacitive technology exists and can be adapted to the sensor to provide distance assessments by arranging a serial array of capacitive strips at predefined or known distances (preferable equal in separation) along the excursion line.

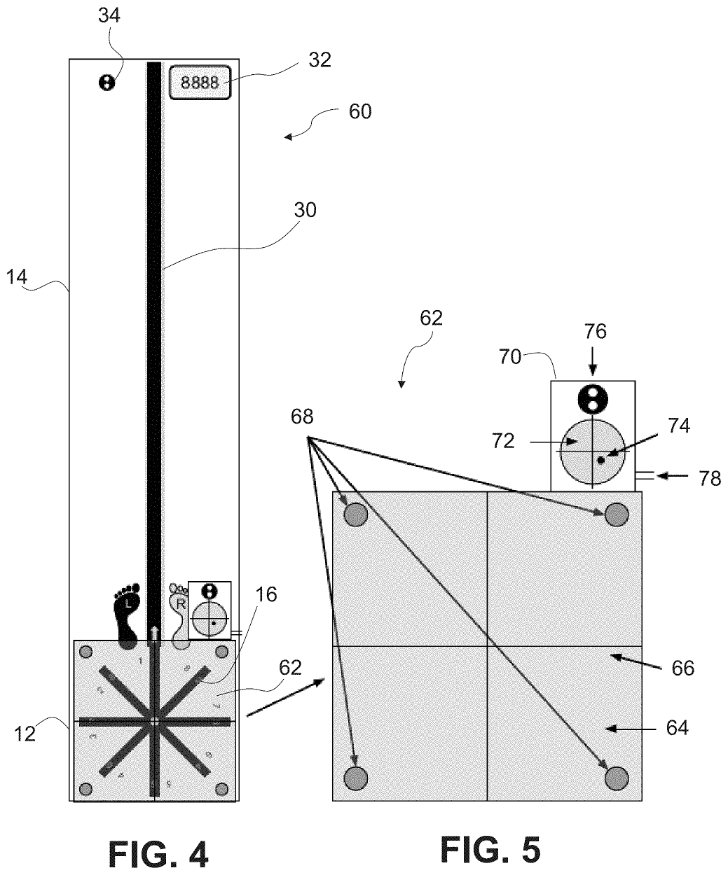

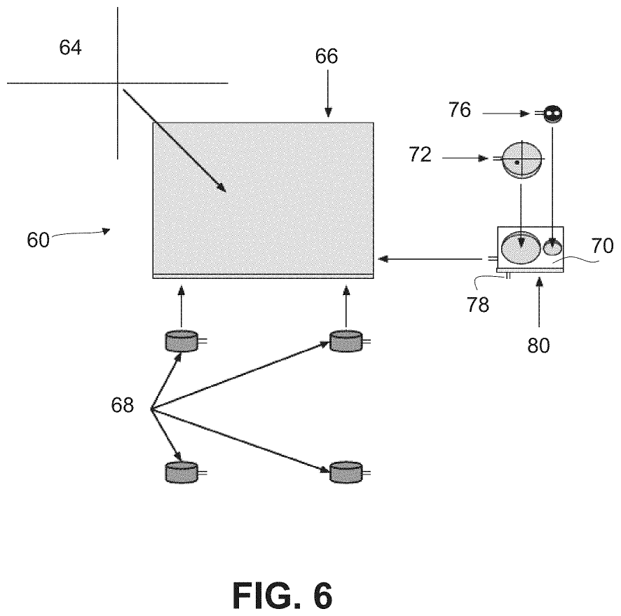

Referring to FIGS. 4 to 6, an alternate balancing test apparatus is shown generally at 60. Balancing test apparatus 60 has all of the features of the balancing test apparatus 10 but it further includes a center-of-gravity platform 62. In the embodiment shown herein the center-of-gravity platform 62 includes a transparent stance surface 64, a stance centering line 66 and at least one load cell 68. In the embodiment shown herein the center-of-gravity platform 62 is a separate structure that is independent of the board and used when necessary. In the embodiment shown herein there are four load cells 68 which are positioned in the corners of the transparent stance platform 66. The load cells 68 are operably connected to a center of gravity display 70. The center of gravity display 70 includes a representation of an allowable zone 72 and a subject center of gravity indicator 74. The subject center of gravity indicator 74 moves responsive to the weight distribution on the subject stance foot as determined from the data received from the load cells 68. The center of gravity display 70 also includes a center of gravity speaker 76 and an external connection port 78. The center of gravity display 70 has a display housing 80.

It will be appreciated by those skilled in the art that the center of gravity speaker 76 could be integrated into the speaker 34. Similarly, the center of gravity display 70 could be integrated into display 32.

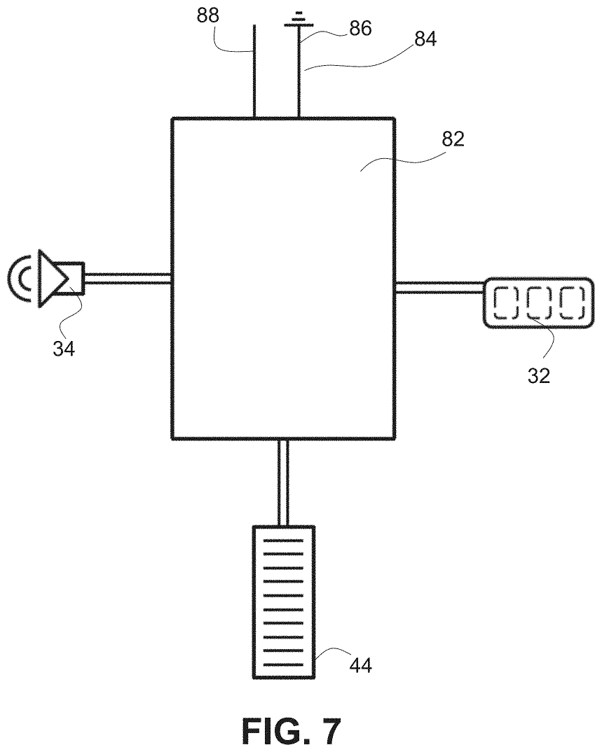

Referring to FIG. 7, a microcontroller 82 is operably connected to the haptic distance sensor 44, the display 32 and the speaker 34. The microcontroller 82 is operably connected to a power source 84. In the embodiment shown herein the power source 84 includes a ground 86 and a voltage source 88. It will be appreciated by those skilled in the art that the power source may be a battery or a conventional plug that is intended to be plugged into a wall socket.

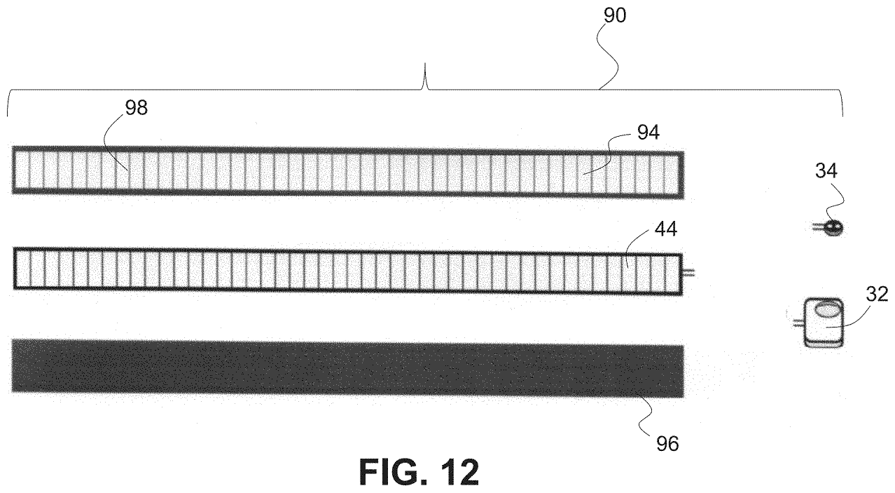

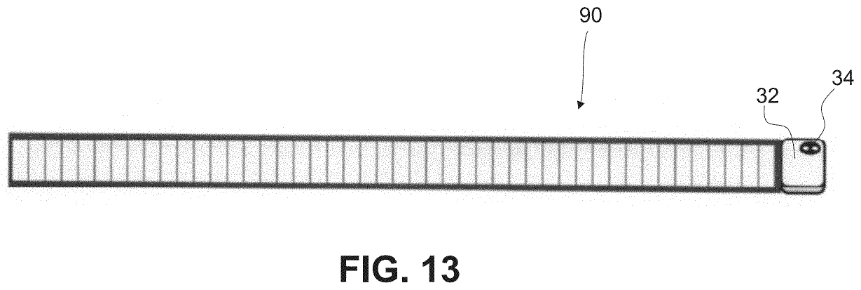

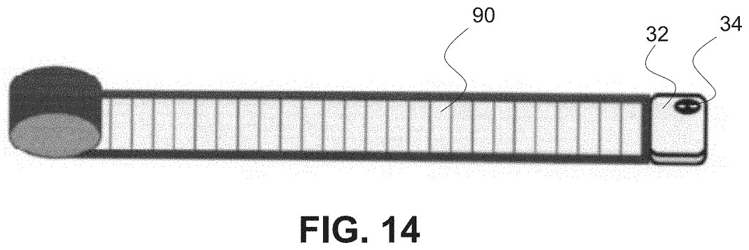

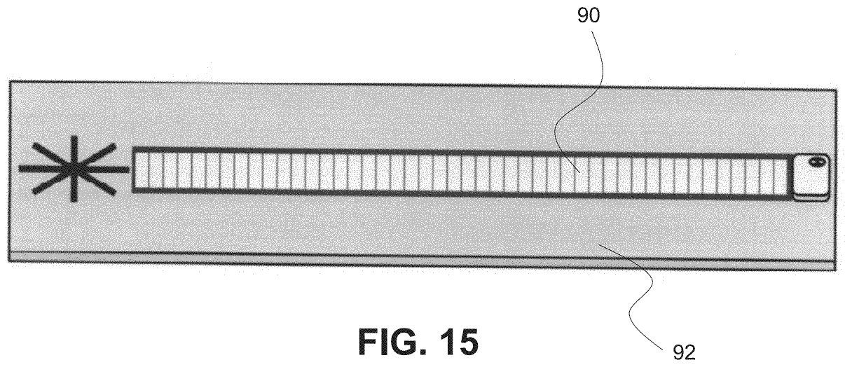

Referring to FIGS. 12 to 15 a flexible sensing strip is shown generally at 90. The flexible sensing strip 90 may be used in association with a prior art Star Excursion Balance Test mat 92 as shown in FIG. 15.

Flexible sensing strip 90 includes an elongate distance sensor 44, a distance display 32 and a speaker 34 as described above. The elongate distance sensor 44 is sandwiched between a top surface 94 and a bottom surface 96. In one embodiment the elongated distance sensor 44 is pliable such that it can be rolled up.

In one embodiment the top surface and the bottom surface is water resistant. More particularly the top surface protects the sensor from water and environmental contaminants and is capable of being cleaned regularly. For example the top surface 94 may be made of vinyl. In one embodiment top surface 94 includes a printed pattern 98. The printed pattern may indicate the scaled distance and for example as shown in FIG. 12 the printed pattern 98 is a ruler. In one embodiment the bottom surface 96 includes an adhesive in order to hold the sensor in place. In one embodiment the bottom surface 96 could be composed of a similar material as the top surface 94 to seal the sensor away from contaminants and having an adhesive base to connect the flexible sensor strip 90 to the top surface of a mat.

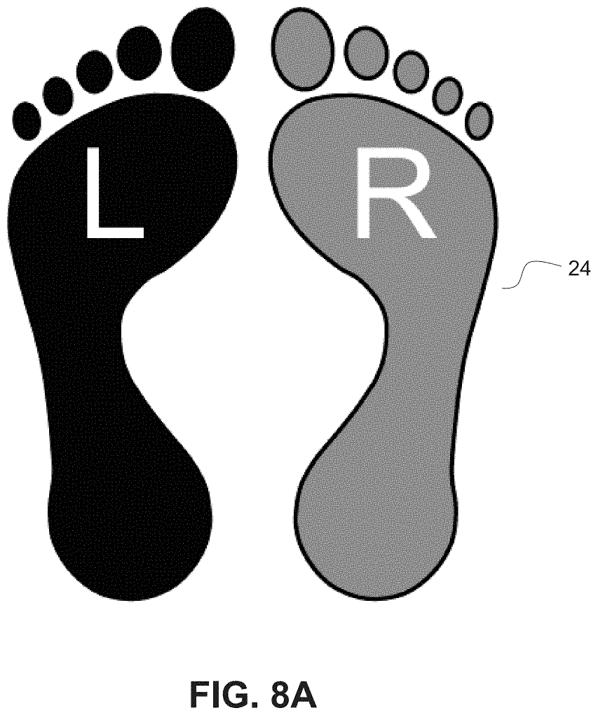

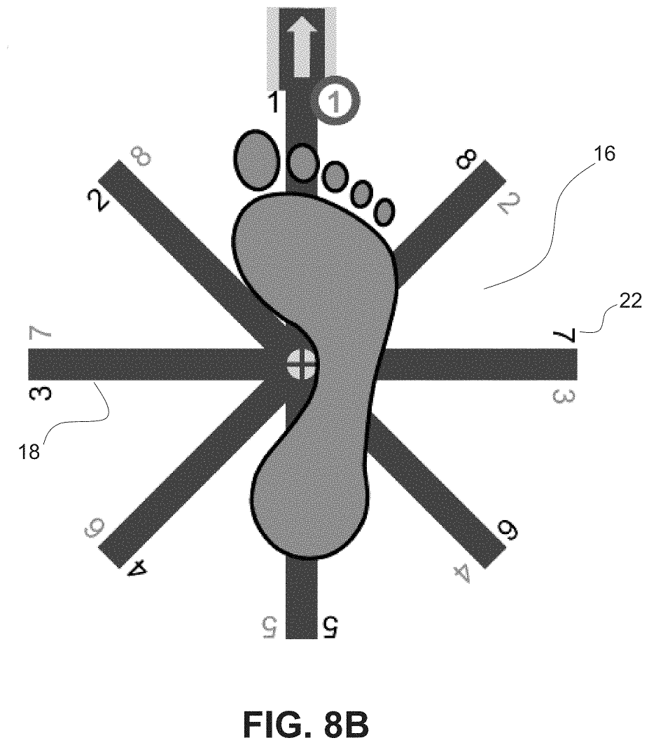

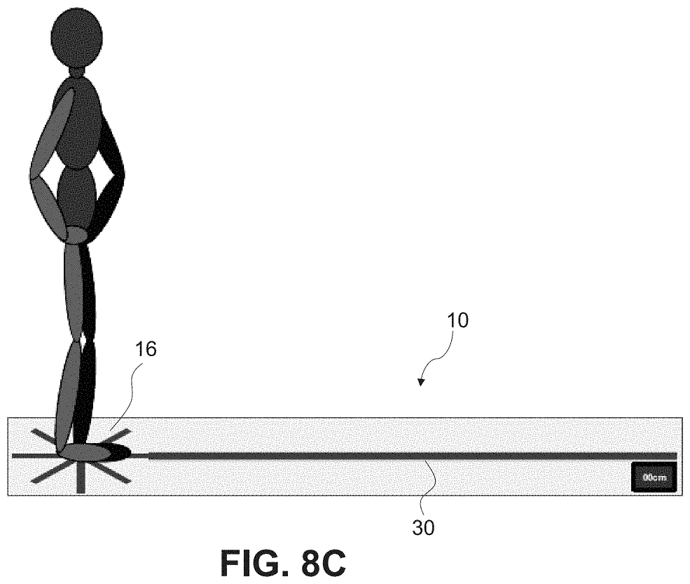

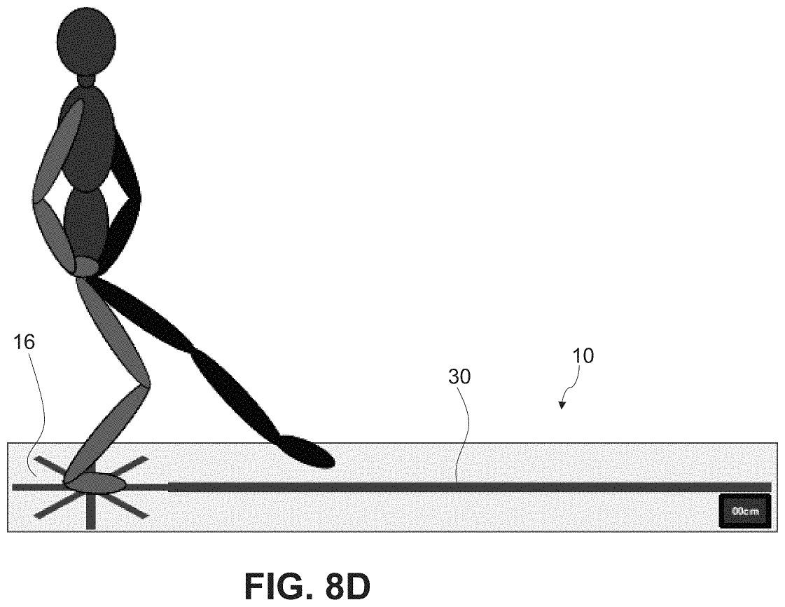

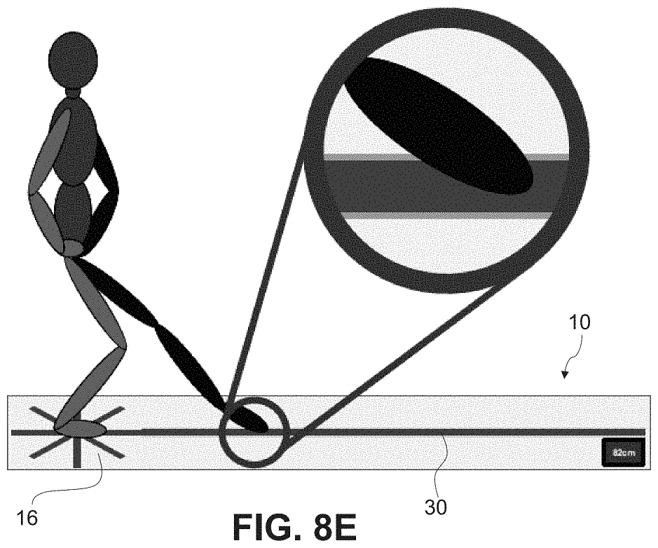

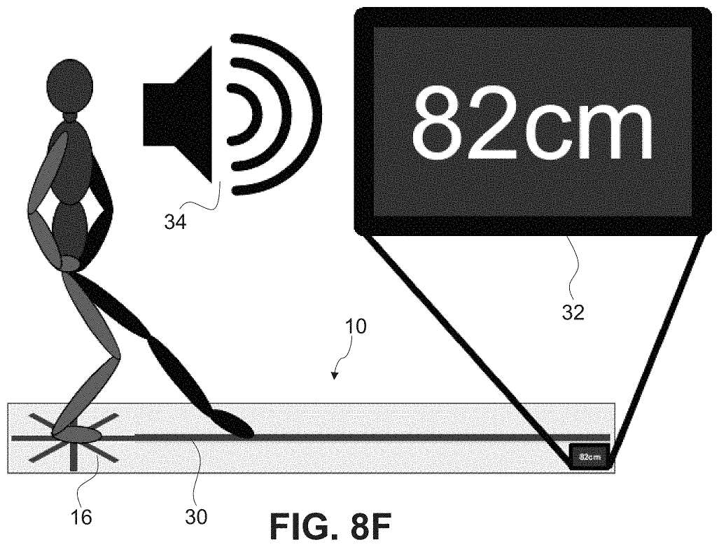

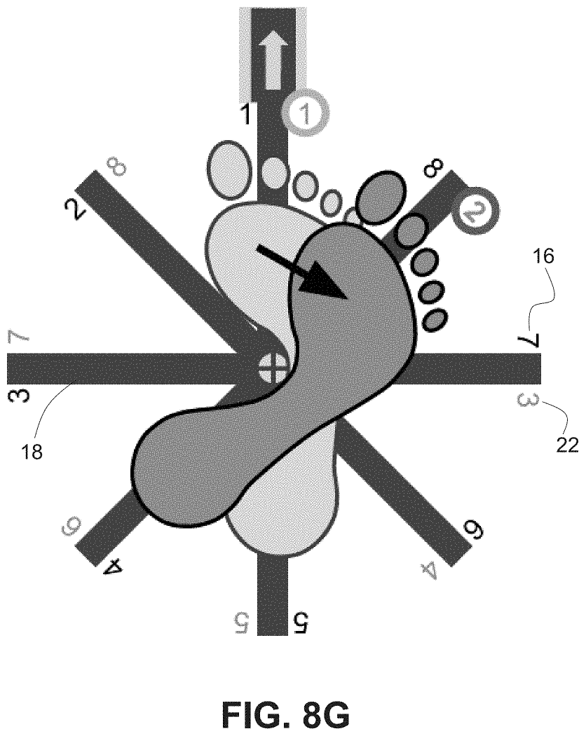

FIGS. 8A to 8G show a pictorial representation of steps used for testing balance of a test subject. Step 1, choose the stance foot as shown in FIG. 8A. Step 2, place the stance foot arch at the central reference point 20 of the orientation guide 16 and the toes along line 1 of the orientation guide 16, as shown in FIG. 8B. Step 3, stand up straight with hands on hip, as shown in FIG. 8C. Step 4, while balancing on the stance foot, reach the excursion foot as far as possible along the elongate reference line 30, as shown in FIG. 8D. Step 5, lightly tap the excursion toe to the elongate reference line 30, as shown in FIG. 8E. Step 6, if the tap is successful, listen for an audible beep and record the excursion length indicated, as shown in FIG. 8F. Step 7, rotate the stance foot to the next numbered position for the stance foot and repeat, as shown in FIG. 8G.

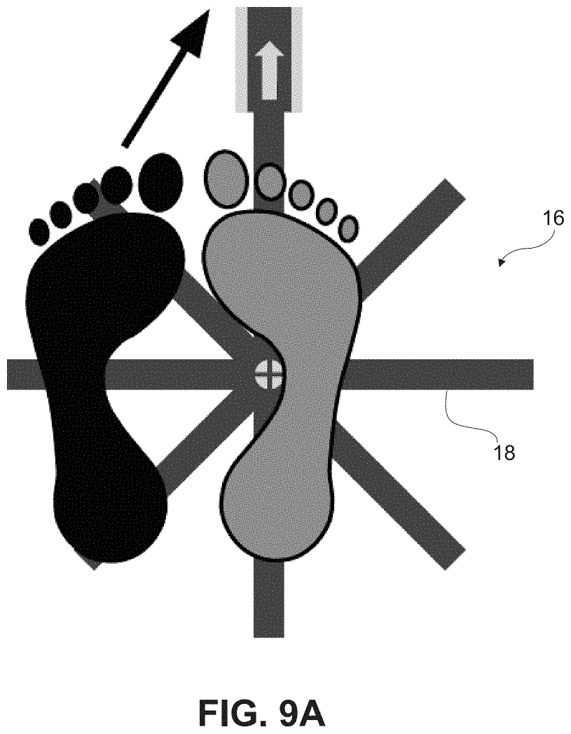

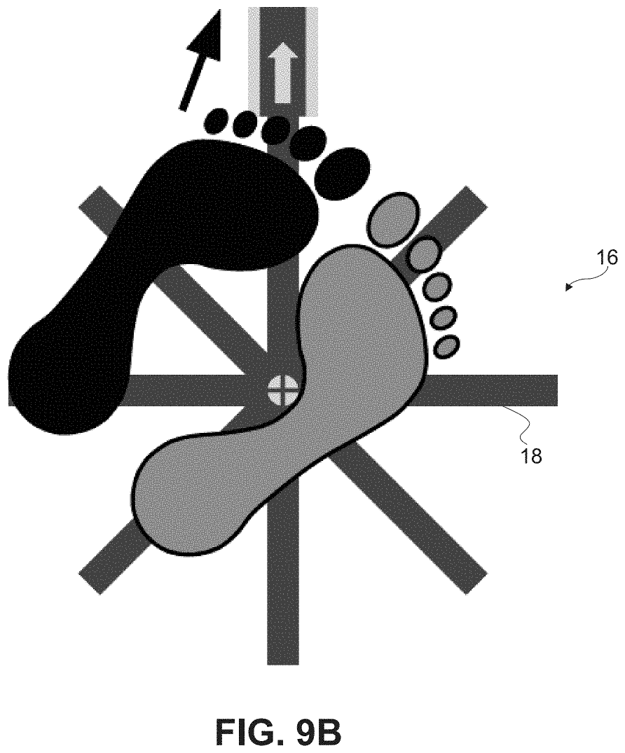

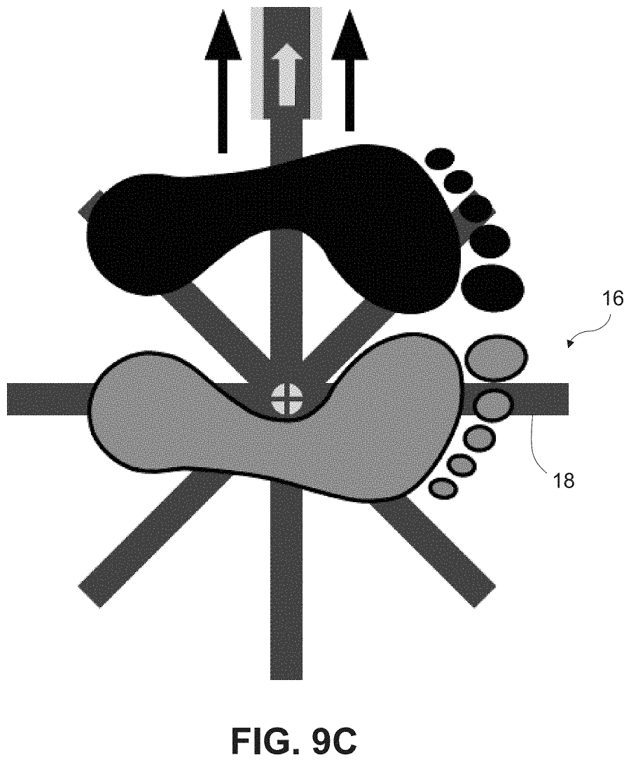

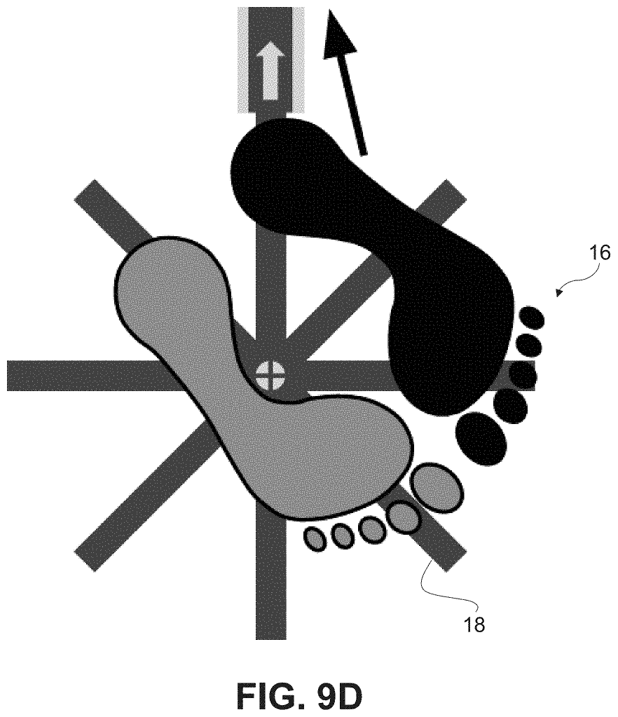

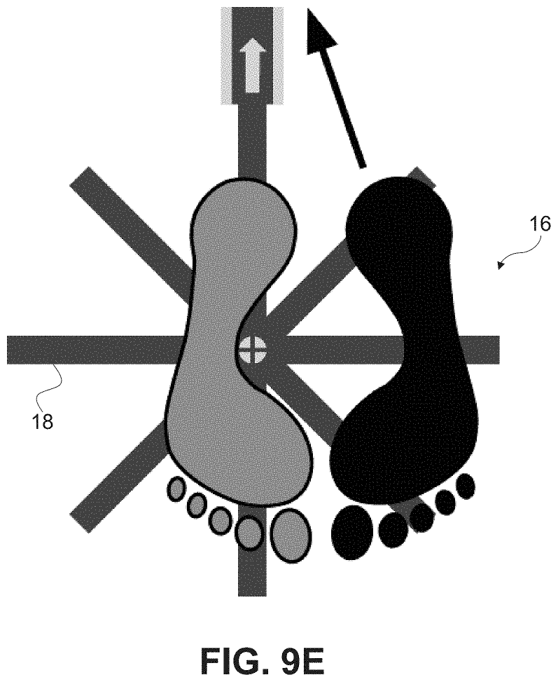

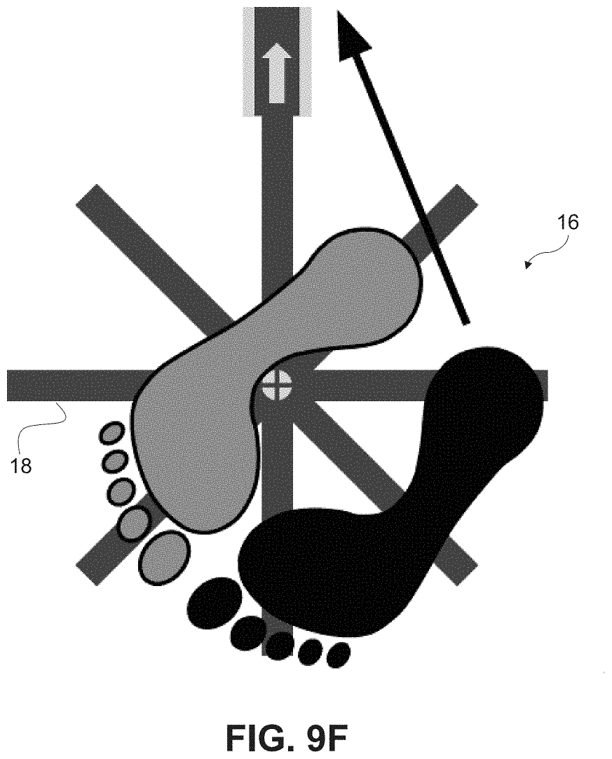

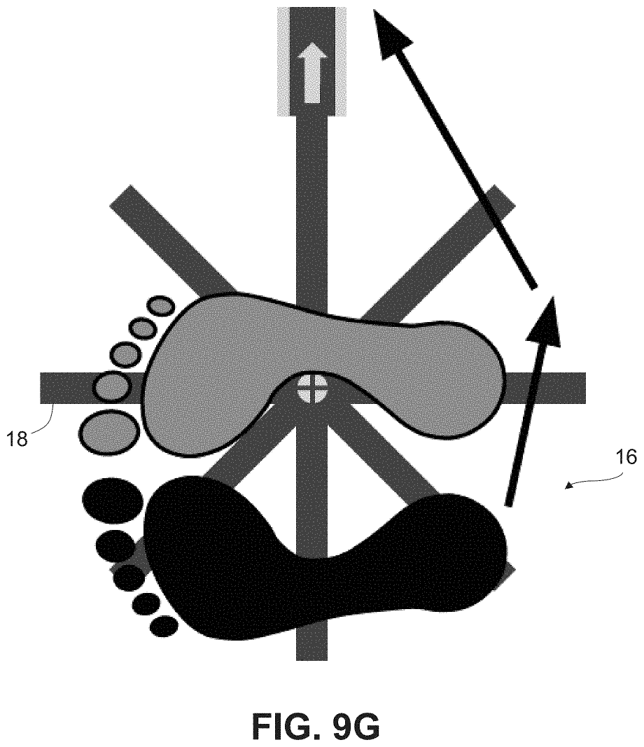

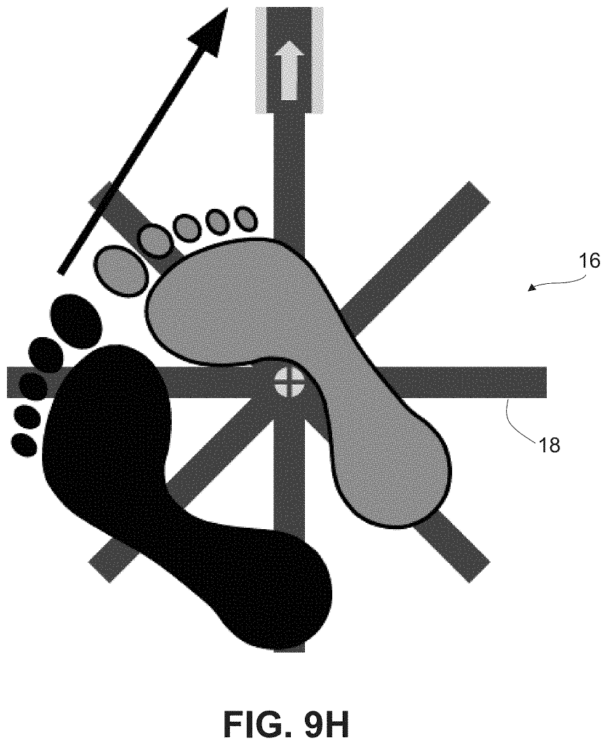

FIGS. 9A to 9H show top views of the stance foot zone 12 showing pictorial representations of the stance foot and the direction of reach for the excursion foot. These pictorial representations are for the right foot as the stance foot. It will be appreciated by those skilled in the art that when the left foot is the stance foot the position of the stance foot and the direction of reach for the excursion foot would be mirror images of what is shown herein. FIG. 9A is the first position with the stance foot being the right foot and being in line with the excursion line in the excursion zone and showing the reach of the excursion foot in front of the stance foot. FIG. 9B is the second position with the stance foot positioned 45 degrees from the first position and the excursion foot extending to the side of the stance foot. FIG. 9C is the third position with the stance foot positioned 90 degrees from the first position and the excursion foot extending to the side of the stance foot. FIG. 9D is the fourth position with the stance foot positioned 135 degrees from the first position and the excursion foot extending backwards past the stance foot. FIG. 9E is the fifth position with the stance foot positioned 180 degrees from the first position and the excursion foot extending behind the stance foot. FIG. 9F is the sixth position with the stance foot positioned 225 degrees from the first position and the excursion foot extending behind the stance foot. FIG. 9G is the seventh position with the stance foot positioned 270 degrees from the first position and the excursion foot extending behind the stance foot. FIG. 9H is the eighth position with the stance foot positioned 315 degrees from the first position and the excursion foot extending backwards past the stance foot.

It will be appreciated by those skilled in the art that the balance testing apparatus 10 or 60 or 90 provide a technological hardware and software solution based on a combination of digital sensors (load cells, capacitive, touch and proximity sensors) to overcome the burden of manual data collection and management. To address the time and effort issue of the current method, the balance testing apparatus 10, 60 may be configured to automatically collect the patient's excursion distance data and populate it in an intuitive and easy to use interface.

The balance testing apparatus 10, 60, 90 may provide instructions such that the patient can be assisted by the practitioner, while both are guided through the assessment with visual and audible cues generated based on the validity of the patient's excursion. The balance testing apparatus 60 may be used to monitor patient balance and record the reach distance along the excursion direction over multiple visits to the clinic. The collected data could then be used to provide the therapist with a baseline assessment and benchmarking, so the treatment progress can be tracked and used to support patient's adherence to prescribed exercises and track intervention effectiveness.

Additionally, the balance testing apparatus 10, 60, 90 reduces the spatial footprint of the prior art SEBT assessment. Balance testing apparatus 10, 60 utilizes a single excursion direction with the patient changing the orientation of their stance foot; as opposed to the standard embodiment of the SEBT where the patient's stance foot is stationary and the excursions are performed in eight separate directions all around the patient's body. Changing the orientation of the stance foot, as opposed to the excursion foot reduces the spatial footprint of the assessment to −10% of its former area as best seen in FIG. 1.

The balance testing apparatus 10, 60, 90 can automate the distance measurement of the SEBT assessment thus allowing the practitioner to solely focus on the patient's postural accuracy. The balance testing apparatus 10, 60 facilitates direct data collection and benchmarking so the treatment and recovery progress could be tracked and used to support patient's adherence to prescribed exercises and track intervention effectiveness.

Generally speaking, the systems described herein are directed to balancing test apparatus. Various embodiments and aspects of the disclosure are described in the detailed description. The description and drawings are illustrative of the disclosure and are not to be construed as limiting the disclosure. Numerous specific details are described to provide a thorough understanding of various embodiments of the present disclosure. However, in certain instances, well-known or conventional details are not described in order to provide a concise discussion of embodiments of the present disclosure.

As used herein, the terms, “comprises” and “comprising” are to be construed as being inclusive and open ended, and not exclusive. Specifically, when used in the specification and claims, the terms, “comprises” and “comprising” and variations thereof mean the specified features, steps or components are included. These terms are not to be interpreted to exclude the presence of other features, steps or components.

As used herein, the term “exemplary” means “serving as an example, instance, or illustration,” and should not be construed as preferred or advantageous over other configurations disclosed herein.

As used herein the “operably connected” or “operably attached” means that the two elements are connected or attached either directly or indirectly. Accordingly the items need not be directly connected or attached but may have other items connected or attached therebetween.

As used herein, the terms “about” and “approximately” are meant to cover variations that may exist in the upper and lower limits of the ranges of values, such as variations in properties, parameters, and dimensions. In one non-limiting example, the terms “about” and “approximately” mean plus or minus 10 percent or less.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result.

What is claimed is:

1. A balance testing apparatus for testing balance of a test subject having a stance foot and an excursion foot, comprising:
a mat having a stance foot orientation guide for receiving the stance foot;
at least one elongate reference line extending radially from the stance foot orientation guide; and
an electronic measuring device for measuring the distance between the stance foot and the excursion foot when the excursion foot is on or near the reference line.

2. The balance testing apparatus as claimed in claim 1 wherein the electronic measuring device is a haptic distance sensor that is located in registration with the at least one elongate reference line.

3. The balance testing apparatus as claimed in claim 1 wherein the electronic measuring device is one of a lidar, capacitive, resistive, ultrasonic, time-of-flight or pressure or temperature sensitive device that measures distance.

4. The balance testing apparatus as claimed in claim 1 further including an electronic data storage device and a visual display operably connected to the electronic measuring device for storing and displaying the measured distance.

5. The balance testing apparatus as claimed in claim 4 wherein the visual display displays diagrams to help guide the subject through the testing responsive to the subject position in the testing.

6. The balance testing apparatus as claimed in claim 4 wherein the electronic data storage device is any one of a mobile device, a personal computer, a tablet, the cloud or a combination thereof.

7. The balance testing apparatus as claimed in claim 1 further including an audio device operably connected to the electronic data storage device.

8. The balance testing apparatus as claimed in claim 6 wherein the audio device provides audio instructions.

9. The balance testing apparatus as claimed in claim 7 wherein the audio instructions are responsive to information collected from the electronic measuring device.

10. The balance testing apparatus as claimed in claim 1 wherein the stance foot orientation guide includes a central reference point for indicating the position of the stance foot.

11. The balance testing apparatus as claimed in claim 9 wherein the stance foot orientation guide further includes a plurality of radial spokes.

12. The balance testing apparatus as claimed in claim 10 wherein the plurality of radial spokes are 8 equally spaced radial spokes.

13. The balance testing apparatus as claimed in claim 9 wherein the stance foot orientation guide is colour coded to differentiate between the position for a right stance foot and a left stance foot.

14. The balance testing apparatus as claimed in claims 9 wherein the stance foot orientation guide further includes a plurality of LED lights.

15. The balance testing apparatus as claimed in claim 14 wherein the plurality of LED lights are activated responsive to the position of the stance foot to indicate the next orientation for the stance foot.

16. The balance testing apparatus as claimed in claim 1 further including a center of gravity platform.

17. The balance testing apparatus as claimed in claim 16 wherein the center of gravity platform includes a transparent surface that is positioned over the orientation guide.

18. The balance testing apparatus as claimed in claim 16 wherein the center of gravity platform is an integral part of the mat.

19. The balance testing apparatus as claimed claims 16 wherein the center of gravity platform includes at least one load cell for determining the center of gravity of the stance foot of the subject.

20. The balance testing apparatus as claimed in claim 19 wherein the at least one load cell is a plurality of load cells.

21. The balance testing apparatus as claimed in claim 16 wherein the center of gravity platform determines the weight of the subject.

22. The balance testing apparatus as claimed in claim 21 wherein the center of gravity platform determines the percentage of the weight of the subject that is offloaded when the excursion foot of the subject touches the ground.

23. The balance testing apparatus as claimed in claim 1 wherein the balance testing apparatus is collapsible.

24. A method of balance testing, for a test subject having a stance foot and an excursion foot using the balance testing apparatus of claim 1 , comprising the steps of:
a) measuring a first predetermined parameter having the stance foot in a first predetermined position;
b) storing the data;
c) repeating steps a) and b) with the stance foot in a different predetermined position; and
d) evaluating the data to obtain a balance test protocol score.

25. The method of balance testing as claimed in claim 24 further including the step of determining the center of gravity on the stance foot in the different positions during the plurality of step a).

26. The method of balance testing as claimed in claim 24 further including the percentage of the weight of the subject that is offloaded when the excursion foot of the subject touches the ground.

27. The method of balance testing as claimed in claim 24 wherein the balance test apparatus includes a program that provides instructions to the test subject responsive to the location of the stance foot.

28. The method of balance testing as claimed in claim 27 wherein the balancing testing apparatus includes LED lights and the lights indicate the orientation of the stance foot.

29. The method of balance testing as claimed in claim 27 wherein pre-recorded audio instructions are provided responsive to the location of the stance foot.

30. A flexible sensing strip for use in association with a balance testing mat for testing balance of a test subject having a stance foot and an excursion foot, comprising:
an electronic measuring device for measuring the distance between the stance foot and the excursion foot when the excursion foot is on or near the reference line having an elongate distance sensor;
a display operably connected to the elongate distance sensor; and
a speaker operably connected to the elongate distance sensor.

31. The flexible sensing strip as claimed in claim 30 elongate distance sensor is sandwiched between a top surface and a bottom surface.

32. The flexible sensing strip as claimed in claim 31 wherein the top surface and bottom surface are water resistant.

33. The flexible sensing strip as claimed in claim 32 wherein the bottom surface includes an adhesive.

34. The flexible sensing strip as claimed in claim 30 wherein the distance sensor is a haptic distance sensor.
